(19) United States
(12) Patent Application Publication
(10) Pub. No.: US 2004/0152780 A1
(43) Pub. Date: Aug. 5, 2004

(54) Process for the preparation of amorphous cilastatin sodium

(76) Inventors: Yatendra Kumar, Gurgaon (IN); Om Dutt Tyagi, Gurgaon (IN); Amit Rohtagi, Delhi (IN)

Correspondence Address: Jayadeep R Deshmukh, Ranbaxy Pharmaceuticals Inc, Suite 2100, 600 College Road East, Princeton, NJ, 08540, US

(21) Appl. No.: 10/478,081

(22) PCT Filed: May 17, 2002
(86) PCT No.: PCT/IB02/01696

(30) Foreign Application Priority Data
May 18, 2001 (IN) 593DEL2001

(51) Int. Cl. A61K 31/198

(52) U.S. Cl. 514/562; 562/506

(57) ABSTRACT

The present invention relates to a cost effective and industrially advantageous process for the preparation of amorphous cilastatin sodium.

### FIELD OF THE INVENTION

The present invention relates to a cost effective and industrially advantageous process for the preparation of amorphous cilastatin sodium.

### BACKGROUND OF THE INVENTION

Cilastatin sodium is the sodium salt of a derivatized heptenoic acid. Chemically, it is [R-[R*,S*-(Z)]]-7-[(2-amino-2-carboxyethyl)thio]-2-[((2,2-dimethylcyclopropyl)carbonyl]amino-2-heptenoic acid monosodium salt and has the structural formula I.

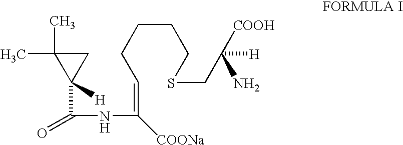

The prototype carbapenem antibacterial agent imipenem, having structural formula II,

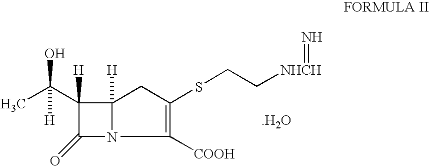

has a very broad spectrum of anti-bacterial activity. It is co-administered with a renal dehydropeptidase inhibitor, cilastatin, in order to prevent its renal metabolism in clinical use. Imipenem/cilastatin sodium combination is a potent broad spectrum antibacterial agent for intramuscular administration. It is an effective monotherapy for septicaemia, neutropenic fever and intra abdominal, lower respiratory tract, genitourinary, gynaecological, skin and soft tissue, and bone and joint infections. In these indications, imipenem/cilastatin generally exhibits similar efficacy to broad spectrum cephalosporins and other carbapenems.

Ciltastatin sodium is disclosed in U.S. Pat. No. 5,147,868, which describes a lyophilization technique to obtain amorphous cilastatin sodium. There is no other prior art reference which describes a method other than lyophilization to manufacture amorphous cilastatin sodium. Lyophilization technique is not a satisfactory technique/process to be used on an industrial scale. This requires large volumes of solvent and capital investments for creating technical infrastructure for lyophilization which makes this process highly unattractive from economical point of view and is not suitable for large scale production.

### SUMMARY OF THE INVENTION

It is an object of the present invention to provide a commercially viable process for the production of amorphous cilastatin sodium which process is very convenient to operate on a commercial scale and does not use capital intensive technique of lyophilization.

Accordingly, the present invention provides a process for the preparation of amorphous cilastatin sodium in pure form which comprises recovering cilastatin sodium from a solution thereof which contains an organic solvent, homogeneous mixture of organic solvents, or homogeneous mixture of organic solvents and water, by solvent precipitation.

The solution from which the cilastatin sodium is recovered is obtained either by dissolving crude cilastatin sodium in a solvent, or obtained from the reaction mixture containing already dissolved crude cilastatin sodium. The term “solvent” as used herein includes organic solvent, homogeneous mixture of organic solvents, or homogeneous mixture of organic solvents and water. The cilastatin sodium in amorphous form is recovered by adding a suitable anti-solvent to the sodium or by adding a solution of crude cilastatin sodium dissolved in a solvent into anti-solvent, by solvent precipitation, isolating and drying the product.

Generally, the product can be isolated by any standard method known in the art such as by filtration, centrifugation or decantation. Typically, the product is isolated by filtration when any of the solvents within the scope of the process are used.

In turn, cilastatin sodium is obtained by suspending cilastatin free acid in a solvent particularly in water or methanol and adding a solution of sodium hydroxide in a solvent, preferably in water or methanol to get a clear solution. The clear solution so obtained is concentrated, in case water is used as a solvent, to get a viscous mass containing crude cilastatin sodium. The viscous mass is further dissolved in a solvent, particularly in methanol, which is concentrated under vacuum to remove the traces of water and to get again a viscous mass containing crude cilastatin sodium.

The solvent is selected from a group of solvents which have the property to dissolve cilastatin sodium and includes methanol. Suitable anti-solvent is any solvent in which cilastatin sodium is insoluble and is miscible with the solvent in which cilastatin sodium is dissolved. In the preferred embodiment of this invention, the solvent is methanol and anti-solvent is acetone.

More particularly, the crude cilastatin sodium is dissolved in methanol and acetone is added to the solution so obtained, or by adding the solution so obtained into acetone, at a temperature ranging from 0° C. to 50° C., preferably at 25-30° C. to get a slurry. The slurry is subjected to vacuum distillation to recover some amount of solvent under reduced pressure and the product is recovered by filtration at ambient temperature after addition of fresh anti-solvent acetone.

Filtration is fast and smooth, which is carried out using nutsche filtration or centrifuge filtration. Preferably, nutsche filtration is used on large scale preparation. Filtered material, a semi dry powder which is further dried to remove surface solvents in a vacuum tray drier, tray dryer, fluid bed drier or a rotary vacuum drier to afford amorphous material. Preferably, material is dried in a vacuum tray drier at a temperature ranging from 20° C. to about 80° C. for about 6 hours to 24 hours. More preferably, drying is carried out at 35° C. to about 40° C. for about 8 hours.

Generally, cilastatin sodium is dissolved in a solvent e.g. methanol at the concentration ranging from about 20% w/v to about 80% w/v, preferably at a concentration of about 30% w/v to about 60% w/v at an ambient temperature.

The volume of anti-solvent varies from about 5 times to 100 times the weight input of cilastatin. Preferably, the volume of anti-solvent used is about 20 times to about 60 times the weight input of cilastatin.

Amorphous cilastatin sodium prepared according to the process of the invention, has been characterized by its X-ray diffraction pattern (FIG. 1), which shows the amorphous nature of the product.

### DETAILED DESCRIPTION OF THE INVENTION

The present invention is illustrated by the following examples which are not intended to limit the effective scope of the claims.

### EXAMPLE 1

(A) Preparation of Crude Cilastatin Sodium

To a suspension of cilastatin free acid (15 gm) in water (80 ml) was added 2N aqueous sodium hydroxide at about 25-30° C. to set the pH of about 7.35. The clear solution so obtained was concentrated under vacuum to remove water to yield a viscous mass. The viscous mass so obtained was dissolved in methanol (150 ml) to get a clear solution which was concentrated under vacuum to get a viscous residue.

(B) Preparation of Amorphous Cilastatin Sodium

Dissolved the so obtained crude cilastatin sodium in methanol (30 ml) and added this solution to acetone (300 ml) under stirring. The resulting slurry is concentrated under vacuum to recover about 100 ml of solvent. Added fresh acetone (100 ml) to the slurry and stirred it for about 30 minutes at 20-25° C. Filtered the separated solid, washed it with acetone (75 ml) and dried the product under vacuum at 35-40° C. to yield dry amorphous cilastatin sodium (15.5 gm, chromatographic purity; 98.96%; pH: 6.94).

### EXAMPLE 2

(A) Preparation of Crude Cilastatin Sodium

Suspended cilastatin (5 gm) in methanol (15 ml) and to it was added methanolic solution of sodium hydroxide (prepared by dissolving 0.558 gm of sodium hydroxide in 15 ml of methanol) slowly under stirring to get a clear solution.

(B) Preparation of Amorphous Cilastatin Sodium

Added the resulting solution into acetone (300 ml) under stirring to get a slurry which was stirred for about 30 minutes at 25-30° C. Filtered the separated solid and washed it with acetone (100 ml). Dried under vacuum at 35-40° C. to yield dry amorphous cilastatin sodium (5 gm; chromatographic purity:99%)

While the present invention has been described in terms of its specific embodiments, certain modifications and equivalents will be apparent to those skilled in the art and are intended to be included within the scope of the present invention.

We claim:

1. A process for the preparation of pure cilastatin sodium in an amorphous form which comprises recovering cilastatin sodium from a solution thereof which contains an organic solvent, homogeneous mixture of organic solvents, or homogeneous mixture of organic solvents and water, by solvent precipitation.

2. The process of claim 1 which comprises recovering pure cilastatin sodium in amorphous form by adding an anti-solvent to the solution of cilastatin sodium in a solvent.

3. The process of claim 1 which comprises recovering pure cilastatin sodium in amorphous form by adding the solution of cilastatin sodium to an anti-solvent.

4. The process of claim 1 wherein the solution of cilastatin sodium is obtained by dissolving crude cilastatin sodium in a solvent or obtained directly from the reaction mixture.

5. The process of claim 1 wherein the solvent has the property to dissolve cilastatin sodium.

6. The process of claim 5 wherein the solvent is methanol.

7. The process of claim 2 wherein the anti-solvent is acetone.

8. The process of claim 1 wherein the cilastatin sodium is obtained by reacting cilastatin free acid with sodium hydroxide.

9. The process of claim 8 wherein the cilastatin sodium is obtained by reacting cilastatin free acid suspended in water with aqueous sodium hydroxide.

10. The process of claim 9 wherein the aqueous sodium hydroxide is of 2N concentration.

11. The process of claim 8 wherein the cilastatin sodium is obtained by reacting cilastatin free acid suspended in methanol with methanolic sodium hydroxide.

12. The process of claim 5 wherein the cilastatin sodium is dissolved in a solvent present at a concentration of about 20% w/v to about 80% w/v.

13. The process of claim 12 wherein the cilastatin sodium is dissolved in a solvent present at a concentration of about 30% w/v to 60% w/v.

14. The process of claims 2 or 3 wherein the volume of anti-solvent ranges from about 5 times to 100 times the weight input of cilastatin.

15. The process of claim 14 wherein the volume of anti-solvent ranges from about 20 times to 60 times the weight Input of cilastatin.
